(19) United States
(12) Patent Application Publication
(10) Pub. No.: US 2007/0077654 A1
(43) Pub. Date: Apr. 5, 2007

(54) Platelets from stem cells

(76) Inventors: James A. Thomson, Madison, WI (US); Dong Chen, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/546,052

(22) Filed: Oct. 11, 2006

Related U.S. Application Data
(63) Continuation-in-part of application No. 11/262,582, filed on Oct. 31, 2005.
(60) Provisional application No. 60/623,922, filed on Nov. 1, 2004.
(60) Provisional application No. 60/714,578, filed on Sep. 7, 2005.

(51) Int. Cl. C12N 5/08

(52) U.S. Cl. 435/372

(57) ABSTRACT

Human embryonic stem cells are induced to differentiate first into the hematopoietic lineage and then into megakaryocytes, the cells which generate platelets. The proper in vitro culture of megakaryocytes results in the production and shed of platelets. This makes possible, for the first time, the in vitro production of a human blood factor needed by many patients. The in vitro produced platelets lack the immune functions that are the source of incompatibility with current platelet transfusions and thus can be universally transfused to patients without issues of immune rejection.

### CROSS-REFERENCE TO RELATED APPLICATIONS

This application is a continuation-in-part of U.S. patent application Ser. No. 11/262,582 filed Oct. 31, 2005 which claims priority from U.S. provisional patent applications Ser. No. 60/623,922 filed Nov. 1, 2004 and Ser. No. 60/714,578 filed Sep. 7, 2005.

### STATEMENT REGARDING FEDERALLY SPONSORED RESEARCH OR DEVELOPMENT

To be determined

### BACKGROUND OF THE INVENTION

Stem cells are defined as cells that are capable of a differentiation into many other differentiated cell types. Embryonic stem cells are stem cells from embryos which are capable of differentiation into most, if not all, of the differentiated cell types of a mature body. Stem cells are referred to as pluripotent, which describes the capability of these cells to differentiate into many cell types. A type of pluripotent stem cell of high interest to the research community is the human embryonic stem cell, sometimes abbreviated here as hES or human ES cell, which is an embryonic stem cell derived from a human embryonic source. Human embryonic stem cells are of great scientific and research interest because these cells are capable of indefinite proliferation in culture as well as differentiation into other cell types, and are thus capable, at least in principle, of supplying cells and tissues for replacement of failing or defective human tissue. The existence in culture of human embryonic stem cells offers the potential for unlimited amounts of genetically stable human cells and tissues for use in scientific research and a variety of therapeutic protocols to assist in human health. It is envisioned in the future human embryonic stem cells will be proliferated and directed to differentiate into specific lineages so as to develop differentiated cells or tissues that can be transplanted or transfused into human bodies for therapeutic purposes.

Platelets are an essential blood component for blood clotting. Platelets are a sub-cellular blood constituent, having no nucleus but hosting cell membranes, receptors, enzymes, granules and other cellular processes, so that platelets are capable of responding to several factors in the blood to initiate blood clot formation. Platelet transfusions are indicated when patients suffer large traumatic blood loss, are exposed to chemical agents or high dose radiation exposure and in a variety of other medical circumstances, such as thrombocytopenia, especial after bone marrow ablation to treat patients with leukemia. The short life span of platelets in storage (typically only 5 days by FDA and AABB regulation) causes recurring shortages of platelets on the battlefield and in civilian healthcare systems.

Of all of the cellular components of blood currently stocked for medical purposes, platelets are among the most fragile. There is currently no clinically applicable method for the long term storage of platelets. For modem healthcare institutions, a shelf life of five days for platelets translates to the clinic shelf life of three to four days, after allowing time for testing and shipping. Many blood banks constantly have logistical difficulties keeping platelets fresh and in stock. Reliably supplying platelets to military field hospitals presents even greater difficulties.

In the body, platelets arise from processes, or proplatelets, formed on cells known as megakaryocytes. The differentiation of megakaryocytes from mouse and human adult hematopoietic stem cells has been studied, but the molecular mechanisms of this differentiation are, as yet, unknown. Long term culture of both adult hematopoietic stem cells and megakaryocytes is difficult, which makes the purification and genetic manipulation of these cells almost impossible. No native human megakaryocyte cDNA library exists and no genetic profiles of normal megakaryocytes are available. The in vitro differentiation of mouse embryonic stem cells has been demonstrated to produce platelets, but the biological function of those platelets is yet unproven. Human and mouse platelets differ significantly. Mouse platelets are smaller and exhibit more significant granule heterogeneity as compared to human platelets. The mechanisms of human and mouse release of platelets from megakaryocytes appears to be significantly different.

There is still significant lack of clarity in the understanding of the process of formation of platelets and the budding of platelets from megakaryocytes. The accepted thesis is that a combination of factors, including plasma and endothelial bound membrane factors, megakaryocyte cytoskeletal or organelle rearrangement, and shearing forces from the blood stream, combine to cause final separation of the mature platelets from the proplatelet structure formed on the megakaryocytes. However, this thesis is largely unproven and the formation and separation of platelets from megakaryocytes is still an area of research where much remains to be uncovered.

### BRIEF SUMMARY OF THE INVENTION

The present invention is summarized as a method for the generation of human platelets which includes the steps of culturing human embryonic stem cells under conditions which favor the differentiation of the cells into the hematopoietic lineage; culturing the cells of the hematopoietic lineage into megakaryocytes; culturing the megakaryocytes to produce platelets; and recovering the platelets.

The present invention is also summarized by quantities of human platelets produced in vitro on demand and in therapeutically significant quantities.

It is a feature of the present invention that platelets produced in vitro do not have bound to them factors encountered in the human bloodstream.

Other objects, features and advantages of the present invention will become apparent from the following specification.

### BRIEF DESCRIPTION OF THE SEVERAL VIEWS OF THE DRAWINGS

FIG. 1 shows a flow diagram of platelet production from human embryonic stem (hES) cells.

FIG. 2 (A) shows a time course analysis of megakaryocyte colony formation; and (B) shows the effect of growth factors on megakaryocyte production.

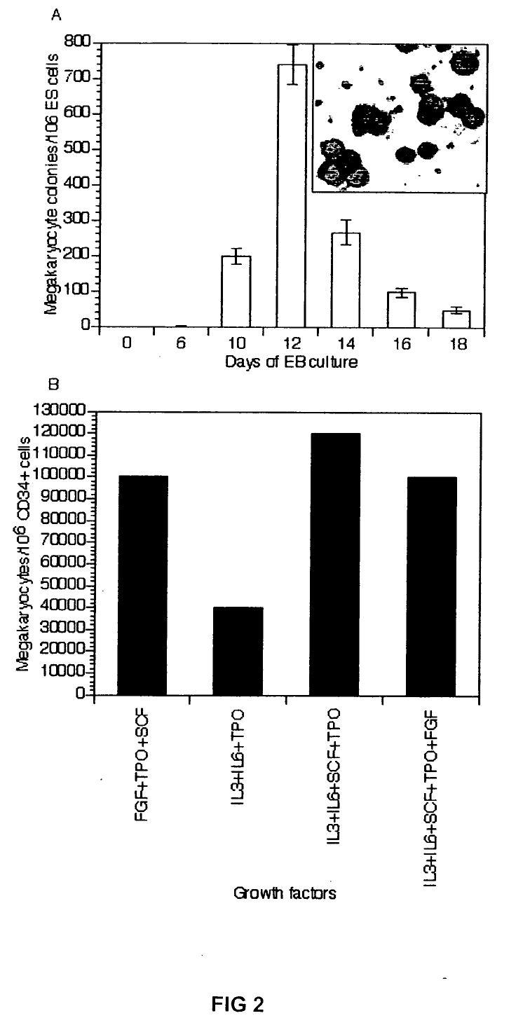

FIG. 3 (A-B) shows images of proplatelets at different magnifications.

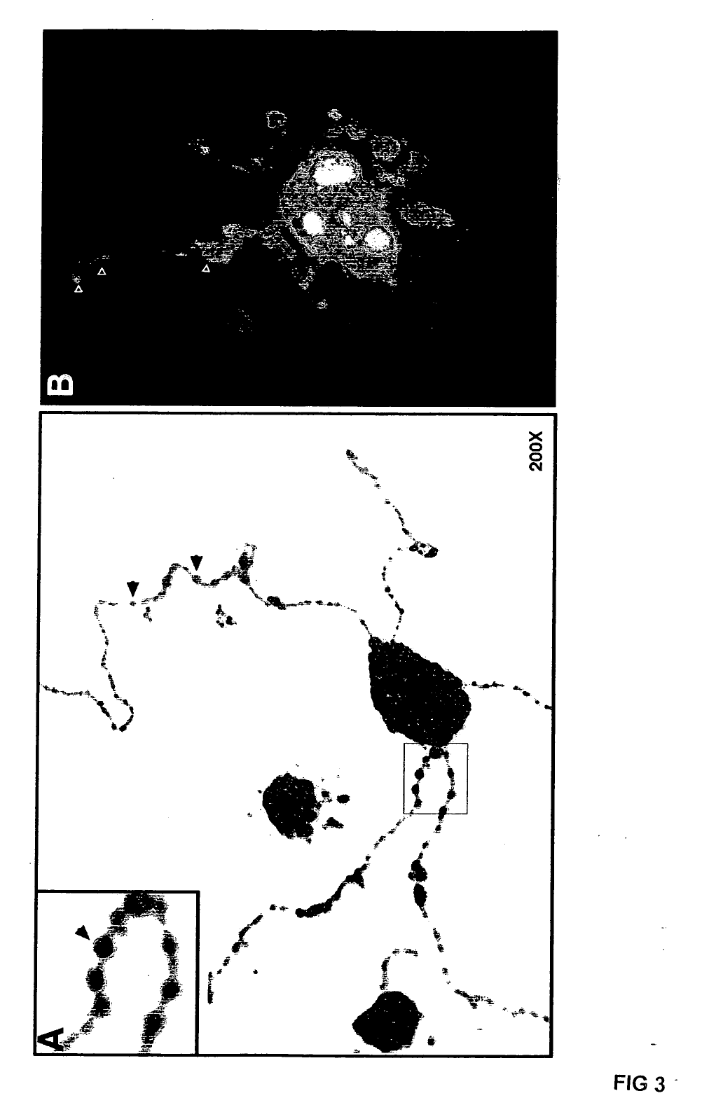

### DETAILED DESCRIPTION OF THE INVENTION

What is contemplated here is the production of platelets by a process of in vitro culture and differentiation beginning with human embryonic stem cells. Human embryonic stem cells (hES cells) are induced to produce megakaryocytes in culture in vitro, and these megakaryocytes are cultured to produce biologically functional human platelets. This process may be thought of as including three major steps or processes. The first major step is the directed differentiation of human ES cells to hematopoietic cells, a differentiation process that, in turn, can be done several ways. Two quite different methods of differentiating hES cells to hematopoietic lineages are described in detail here. In one technique for the hematopoietic differentiation process, human embryonic stem cells (ES cells) are cultivated to form embryoid bodies (EBs), using a previously known technique. The embryoid bodies are cultured so that differentiation of various differentiated cell types can begin, after which the embryoid bodies are disaggregated into a cell suspension in a medium selective for megakaryocyte precursors. With the help of a time course cDNA microarray analysis, we have identified the most optimal time to harvest definitive hematopoietic cells that have the highest hematopoietic potential. The other technique, already demonstrated to be sufficient for the creation of hematopoietic cells, calls for exposure of the human ES cells to stromal cells, an exposure that causes the ES cells to differentiate preferentially to cells of the hematopoietic lineage. The result of either of these processes is a culture of cells that are, to some degree of purity, predominantly ES cell derived hematopoietic cells. We particularly favor the embryoid body approach not only because it does not have contamination from feeder cells of different species, but also it can be performed with a defined serum free media. In the second major part of the process, these hematopoietic cells are then exposed to a selective megakaryocyte formation medium containing growth factors that specifically encourage the formation of megakaryocytes and promote maturation of these cells. Finally these mature megakaryocytes are exposed to platelet formation medium to promote in vitro platelet production. During all these processes animal or human serum and plasma can be avoided.

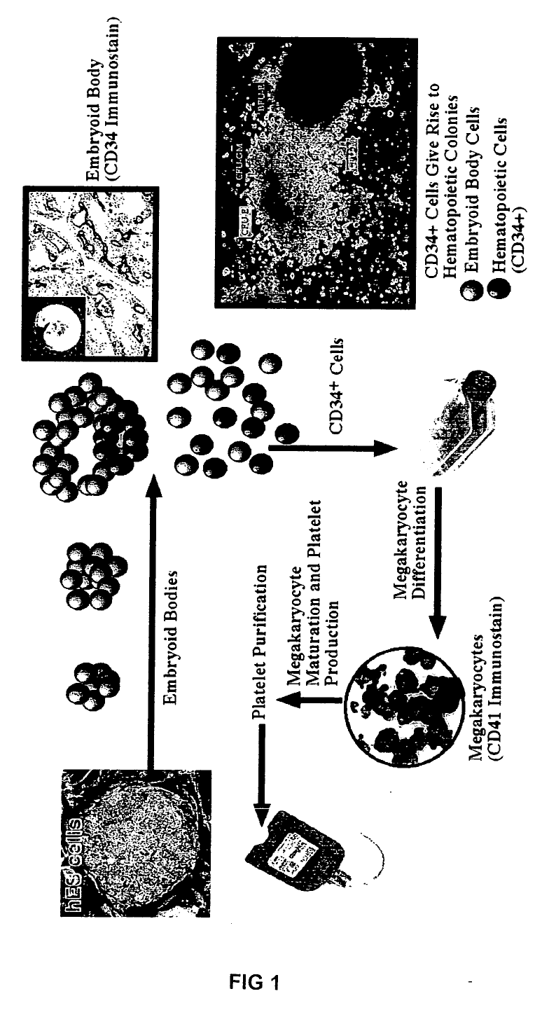

The overall process is illustrated in FIG. 1. Human embryonic stem cells (hES cells) are shown cultured to form embryoid bodies. The embryoid bodies yield CD34+ cells, which are hematopoietic and endothelial precursors. The hematopoietic cells are then differentiated into megakaryocytes, which are CD41+/CD61+, and ultimately platelets are produced.

Platelets are an exemplary target for the production of biological products for human use from hES cells, because platelets carry no chromosomal genetic material. Platelets may be thought of as cytoplasmic fragments of the parental megakaryocytes. Importantly, platelets exhibit both cell surface factors that can carry out adhesion, aggregation and granule secretion. Since the process of platelet maturation and the process of platelet shed from megakaryocytes are both processes that are poorly understood, it was not known if biologically functional platelets could be recovered from in vitro cell cultures derived from human ES cells. Here it is disclosed that platelets can be recovered in useful quantities from such cell cultures.

Importantly, it is also demonstrated here that platelets are capable of being formed and shed from human megakaryocytes in an in vitro cell culture. Given the uncertainty surrounding knowledge of the detailed biology of this process, it was not known previously if this would or could occur in culture. The results here demonstrate that it can and does.

Also, it was unknown before the work described here whether platelets produced by in vitro culture would be functional. Platelets produced in vivo are generated in the presence of human plasma. Since the processes which result in mature platelets are not well understood, it was not known if any interactions with other plasma constituents were required for platelet functionality. Here it is shown that in vitro produced platelets, which have never been exposed to human serum or plasma, are functional by standard platelet tests for functionality.

The present process begins with hES cells, which are by definition undifferentiated cells in culture. It has been previously demonstrated that hES cells can be induced to differentiate into a culture of cells in which cells of hematopoietic lineage predominate. Two different techniques are so far known in the literature for achieving this directed differentiation, and it is envisioned that other techniques will work as well. One known technique calls for the development of embryoid bodies, which are aggregates of hES cells which acquire a three dimensional structure, and that structure seems to encourage differentiation of stem cells into committed progeny lineages. From such embryoid bodies, which produce differentiated cells of a variety of lineages, selective protocols can then be used to isolate cells of the lineage sought, such as cells of hematopoietic lineage. A detailed time course analysis of the hematopoiesis done by us has provided us a genetic profile of these hematopoietic precursors and at the same time we have optimized our protocol to produce the highest yield. The other documented technique involves the co-culture of hES cells with human or non-human stromal cells. Such a co-culture with stromal cells also seems to induce hES cells to produce predominantly hematopoietic cells, but current techniques are based on culture conditions some might seek to avoid.

An intermediate step in the EB method, which has been found to increase the yield of cell of the various hematopoietic lineages, is to fragment the EBs. One of the characteristics of EBs is that the EBs can grow so large as to exceed the ability of the medium to provide oxygen and nutrients to the cells in the center by diffusion. The result can be a necrotic area in the center of the EB, which also causes growth of the EB to stall. It has now been found that by fragmenting the EBs, i.e. by physically chopping the EBs into pieces, one can restart the growth of the EBs which results in more differentiated cells. In our hands, using this technique has resulted in a dramatic increase in the numbers of blood cells recovered from the overall process. Various mechanical devices and systems can be used to assist in this fragmenting or chopping process of the EBs.

Once cells of the hematopoietic lineage are produced, the cells are then cultured to preferentially produce megakaryocytes. This process does not have to be absolute, but culture conditions preferential for megakaryocytes will increase the proportion of megakaryocytes in relation to other blood product precursor cells in the culture. Conditions favorable for the production of immature and mature megakaryocytes include culture of precursor cells cultures with thrombopoeitin (TPO), interleukin 3 (IL3), interleukin 6 (IL6) and stem cell factor (SCF). We also determined that FLT-3 is helpful to the process as well. Immature megakaryocytes can be further expanded if bFGF (basic fibroblastic growth factor) is present. The megakaryocytes obtained by this method are positive for CD41, CD42a, CD42b, CD61, CD62P, CD38, weak CD45, but negative for HLA-DR, CD34, CD117. This immunophenotypic profile is constant with normal mature megakaryocytes. There is a small significant CD45+ population that is exclusively positive for CD14 suggesting that they are mainly monocytes rather than lymphocytes.

Platelet formation and release by megakaryocytes then can be made to occur in culture. While the exact mechanism responsible for release of platelets in vivo is not completely characterized, platelets in cell culture can be made to release from their parental megakaryocytes. We think that four factors that could be potentially crucial. These four factors are shearing force, megakaryocyte-endothelial cell interaction, plasma factors and finally molecular mechanisms in megakaryocytes. Shearing force of the blood can be simulated by physical manipulation of the culture container, as by shaking, rotating or similar process. The role in release actuated by plasma proteins and platelet receptors can be actuated by the megakaryocytes themselves, or factors can be individually added, as needed. Large platelets have been described in certain congenital platelet abnormalities such as Bernard Soulier disease and von Willebrand factor (VWF) disease. We have observed some similarly large platelets in some of our embryoid body derived platelets. If this phenomenon is observed, due to inefficient pinching of platelets from proplatelets caused by the lack of plasma factors such as VWF, this problem can be addressed by the addition of VWF alone or of VWF included in plasma. The experience to date is that none of these plasma factors are needed and that the platelets will readily separate from the megakaryocytes in culture. It has been hypothesized that cGMP can promote platelet formation from neoplastic megakaryocytes. cGMP can be activated by nitric oxide. We have found that the addition of GNSO, a nitric oxide releasing compound, can quickly fragment megakaryocytes into small platelet-like particles in 2 hours. Finally, a by-product of this process is a relatively pure culture of endothelial cells. We are testing to determine if endothelial cells can also help in platelet shedding. By all these techniques, we can significantly increase the efficiency and regularity of platelet formation. To understand the biological mechanism of platelets, we have set up 3D real time fluorescent microscopy to record the platelet release with and without the presence of plasma. We have been able to record the 3D image of proplatelets and we are currently are in the process of doing time lapse imaging to better monitor this process.

Platelets can then be gathered and packaged. At the final stage of megakaryocyte differentiation on day 12, non-cohesive megakaryocytes can be transferred into the upper well of a multi-well plate with 3 μM pore size filter. Incubation will be carried out in an incubator with gentle shaking and GNSO. Platelets can be collected in the lower chamber, and, if desired, in the presence of human plasma, or VWF and fibrinogen at physiological concentrations. Platelets isolated from this in vitro system can be purified by sequential low-speed centrifugation and re-suspended in citrate buffer as donor platelets. The collected platelets can be further centrifuged at low speed (3000 g for 30 min) to separate the other debris and then filtered through an appropriately sized filter to rid the preparation of any nucleated cells. The platelet containing product thus produced can feature the platelets concentrated to any desirable concentration. The in vitro produced platelets can be further purified as serum or plasma free products to fit particular clinical needs. All containers can and should be sterilized to decrease the bacterial contamination, a common problem with donor platelets from conventional sources.

The platelets thus produced from in vitro cell culture will be different from those that have previously been available to science or medicine, in that these platelets will not have been exposed to the bloodstream. Platelets produced in vivo in an organism can not be completely separated from plasma using present commercial techniques. As a result, the packaged platelets in current medical use today also carry small quantities of lymphocytes and plasma contaminants that can cause transfusion reactions in some patients. Platelets produced from this in vitro system by differentiation from human ES cells can be produced essentially free of lymphocytes, and the platelets will never have been exposed to serum or plasma. Platelets produced by this in vitro system will carry fibrinogen only if the factor is added in the growth or separation process. Furthermore, our work so far has shown that the ES in vitro derived platelets lack or have low functional HLA-class I antigens, and this again is different from platelets derived from human donors. These in vitro produced platelets should also have low or no ABO antigen and absolutely no Rh antigen. Thus, the platelets produced from this in vitro platelet production system should not need to be matched for the recipient, but instead can be used universally.

The white blood cells that one desires to avoid in the production of platelets are the constituents of the immune systems such as T cells and B cells. The post-transfusion purpura or refractory thrombocytopenia observed in some patients is generally caused by antibodies from the recipient acting against donor HLA class I antigens on the membranes of the transfused platelets. The most important cause of this phenomenon is the contamination from lymphocytes which is inherent in current techniques to isolate platelets from donor blood. However, the platelets produced by this in vitro system should be entirely free of lymphocytes in general and T cells and B cells in particular.

Another issue is that platelets isolated from human donors also inevitably carry immunoglobulin molecules from the donor in the plasma. The contamination of the plasma limits the universal usage of platelet transfusion to mis-matched recipients. Platelets produced in vitro from ES cells will not have been exposed to plasma IgGs and will thus be free of them, which makes them a universal transfusible agent when compared to donor platelets. The lack of donor plasma, lymphocytes, ABO and Rh antigen, and the lowered risk of infectious contamination, will make platelets produced in vitro medically and scientifically more adaptable as well as readily distinguishable from platelets produced by conventional separation techniques.

### EXAMPLES

Hematopoietic Precursors from Embryoid Bodies

Embryoid body (EB) formation is a method that has been used to study hematopoietic differentiation of both mouse and human ES cells. However, unlike mouse ES cells, human ES cells in a single cell suspension fail to efficiently form embryoid bodies. Instead, to form embryoid bodies from human ES cells, intact colonies of human ES cells cultured on mouse embryonic fibroblasts (MEFs) were digested for 5 min by 0.5 mg/ml dispase to form small cell clusters. These cell clusters were then allowed to further aggregate in serum-containing stem cell cultivation medium (15% FCS). Easily distinguishable cell masses, embryoid bodies start to form after 6 days of culture with 50% single cells that fail to participate in cell masses and undergo apoptosis. After 12 days of culture, the embryoid bodies resembled the early embryonic structure of the yolk sac. Sectioning of the embryoid bodies followed by subsequent immuno-labeling the sections by a CD34 antibody revealed the histological features of yolk sac. Non-adhesive hematopoietic precursor cells were found to be present in the lumen of small vessels and the endothelial lining, as revealed by the cells being CD34 positive. The embryoid bodies were then treated by a 30-minute collagenase (1 mg/ml) and 5 minute trypsin digestion (0.05% Trypsin/0.53 mM EDTA) at 37° C. Approximately 10⁵ embryoid body-derived cells containing primary hematopoietic precursor cells were plated in methylcellulose cultures (Stem Cells Inc. Canada) and cultured for 10-12 days. Erythrocytes and megakaryocytes colony forming units (CFUs) were then detected by their native red color or immunolabeling with monoclonal anti-CD41 or CD61 antibodies. Definitive hematopoietic precursor cells that can give rise to macrophage and granulocyte colonies formed at day 12. This activity represents the first wave of primary and definitive hematopoiesis. Separately, we now also established a serum free embryoid body culture system free of both animal and human serum, and so it is believed that serum is not a requirement for the initiation of embryoid bodies, even though it was used in the work described above.

In Vitro Expansion of Embryoid Body-Derived Megakaryocyte Precursors

After 12 days of embryoid body formation and culture, a single cell suspension culture was made by a 30-minute collagenase (1 mg/ml) and 5 minute trypsin digestion (0.05% Trypsin/0.53 mM EDTA) at 37° C. of the resulting cell cultures. CD34+ cells were separated from other EB cells, since they can interfere with hematopoiesis. CD34+ cells were cultured on a poly-HEME surface in the presence of thrombopoietin (TPO), interleukin 3 (IL3), interleukin 6 (IL6), FLT-3, and stem cell factor (SCF), all factors chosen to specifically promote megakaryocyte differentiation and proliferation. A yield of 10⁵ CD41+ megakaryocytes per 10⁶ starting ES cells was obtained (n=6). Interestingly, when plated in collagen-based semisolid matrix, these megakaryocytes formed extremely long processes with bead-like structures representing proplatelets. Such long structures have not previously been reported when human adult hematopoietic stem cells or mouse ES cells were used in attempts to generate megakaryocytes. Small CD41 positive cell fragments, identified as released platelets, were detected to be present close to the megakaryocytes. By flow cytometry, we found these megakaryocytes are positive for CD31, CD41, CD42a, CD42b, CD61, CD38, CD45 (weak) and CD62P, but negative for CD34, CD117, beta-microglobulin (HLA class I beta subunit) and HLA-DR. This phenotypic profile is consistent with normal human mature megakaryocytes.

Differentiation of Megakaryocytes from Human ES Cells on Stromal Layers

The OP9 stromal cell line is a cell line established from newborn calvaria op/op deficient mice that has been used to support mouse hematopoiesis. The op/op mouse carries a mutation in the coding region of the macrophage colony-stimulating factor (M-CSF) gene. Results of differentiation of human ES cells to hematopoietic lineage using the OP9 system were similar to the method of differentiation of human ES cells by embryoid body formation, but the stromal cell method usually gave a higher yield of more mature precursor cells. Briefly, human ES cells were seeded on confluent OP9 stromal cells and then cultured in alpha-MEM medium supplemented with 20% fetal bovine serum (FBS). Differentiation was started with 10⁵ ES cells per well of a six-well plate or 8×10⁵ cells in a 10 cm² culture dish. After 6 days of culture, the ES cells differentiated into hematopoietic progenitors, as indicated by the emergence of CD34+ cell surface markers on the cells. For differentiation into megakaryocytes, the cells were trypsinized on day 6 (0.05% Trypsin/0.53 mM EDTA at 37° C./5% CO₂) for 5 minutes and passed onto fresh confluent OP9 cells in the same culture medium containing 10 ng/ml TPO. After an additional 8 days of culture, megakaryocytes could start to be seen by visual inspection. About 30% of cells in the supernatant of the culture were megakaryocytes, as confirmed by CD41 immuno-staining. These megakaryocytes are multinucleated but without the significant long processes that were seen in embryoid body-derived megakaryocytes. These megakaryocytes are believed to be definitive megakaryocytes that closely resemble the adult megakaryocytes. Interestingly, during the culture, there was no sign of platelet formation which is quite different from the murine system. It is likely that OP9 cells can promote and support megakaryocyte differentiation and proliferation, but can not support platelet formation. This is another indication that the mechanisms of platelet formation in mouse and human are different, even though some of the mechanisms of megakaryocyte differentiation and proliferation are similar.

Megakaryocyte Proliferation, Maturation and Purification

Precursor megakaryocytes derived by either of the above methods have demonstrated the ability to proliferate and even engraft in adult recipient mice. As a next step in the process, we used bFGF to further proliferate immature megakaryocytes and at the same time halt the megakaryocyte maturation. Estimating that each megakaryocyte can generate 2000 platelets, 10⁷ human ES cells (two 6-well plates) would generate 10⁶ megakaryocytes and subsequently about 2×10⁹ platelets, which represents approximately 1/20 unit of platelets (>5.5×10¹⁰ platelets per unit). So, at this estimated efficiency, to make 1 unit of human platelets would require 20 to 40 T75 flasks of human ES cells. This may or may not be economically attractive at this yield, but it is clearly in the range of what a single technician can already support, particularly if using automated equipment.

Alternative Techniques to Direct Differentiation

The embryoid body system has a lower than desired efficiency of making hematopoietic stem cells, due to the fact that the majority of the cells are yolk sac cells. However, this system is superior to the OP9 co-culture system since the embryoid body system has no murine protein contamination. From our data, we believe that hematopoietic differentiation is still best accomplished in the EB system as opposed to the co-culture system with stromal cells. To get more definitive hematopoietic cells and make the process more efficient, we plan to prolong the EB culture. We have tried to mechanically dissect or fragment the EBs into smaller fragments and continue the culture hoping that the micro-environment will continue to support blood island differentiation. Our preliminary observation suggests that dissected EBs can survive and continue to grow following this dissection. This will be the first attempt to push the differentiation further in the EB system. Even if definitive blood islands can not form, significant increase in the number of blood islands may be achieved. Addition of growth factors in the EB culture such as VEGF and SCF will also be tested.

Improved Platelet Release and Maturation

Although we have already observed platelet formation in multiple systems including collagen matrix, OP9 and polyHEME we want to better understand the mechanism of platelet release so that the process can be optimized. In order to achieve platelet formation we will culture 10³˜10⁴ mature megakaryocytes in the presence of TPO, human plasma, and nitric oxide. The platelets will be labeled with anti-CD41 antibody and counted by flow cytometry. The shape of the platelets will be examined by microscopy, including electron microscopy. True platelets should be discoid without processes or attachment with other platelets. Larger or linked platelets suggests non-optimal conditions that only support proplatelet formation or embryonic platelet formation. Extracellular matrices such as fibrinogen and fibronectin have been shown to promote megakaryocyte proliferation and maturation. We will use fibrinogen and fibronectin coated plates to culture megakaryocytes to determine their effects on megakaryocyte proliferation and differentiation. As mentioned earlier, the addition of nitric oxide has been found to significantly promote platelet production and/or release from the megakaryocytes.

Testing Platelets

Platelets can be morphologically normal under light microscopy and be immunologically similar to platelets yet still lack the physiological functionality of mature platelets. For this reason, it is important to test the functional characteristics of in vitro produced platelets using normal platelet function tests.

Platelet aggregation, in response to thrombin, ADP, and collagen, is one of the initial steps in forming a blood clot. The stimulant, such as thrombin, triggers a cascade of signal transduction and results in the increased affinity of fibrinogen for its platelet surface receptor glycoprotein IIIbIIIa (CD41). Fibrinogen molecules bridge the adjacent platelets and result in a platelet plug. This aggregation function of platelets was measured for the in vitro produced platelets by two methods. The first method is the tube clot formation assay in which platelets and fibrinogen were incubated in the presence of TRAP (thrombin analog) which acts to activate platelets. The platelets were then spun down on glass slides and stained with antibodies against CD41. With the presence of fibrinogen and TRAP, the in vitro produced platelets formed aggregates that were positively stained by CD41 antibodies. Thus this first test for platelet aggregation yielded a positive result.

The second method used to test platelet aggregation uses flow cytometry. Resting platelets produced from the in vitro system were incubated with fluorescent-conjugated fibrinogen with and without the presence of TRAP. After 5 minutes of incubation, both samples were analyzed by flow cytometry by gating on platelets (CD41+). The right shift of the fibrinogen fluorescent intensity demonstrated increased affinity for fibrinogen among the TRAP activated platelets. Thus both tests demonstrated the competence of the platelets to aggregate.

There are three main types of platelet granules: alpha granules, dense core granules and lysosomes. We tested the alpha release granule release by analyzing the surface presentation of an alpha granule membrane protein P-selectin (CD62P). Platelets were gated by CD41 and right shift of the CD62P upon thrombin activation thus indicating the release of alpha granules. We measured dense core granules release by analyzing ATP release with a commercial luciferase assay developed for that purpose (Promega). We first established the ATP release assay using ATP control, donor platelets and Meg-O1 cells. Then we tested the supernatant from low speed centrifugation of the day 12 megakaryocyte culture with and without thrombin stimulation. The platelets in the supernatant responded to the thrombin stimulation by secreting ATP, resulting in an increased reading indicated dense core particle release.

These tests established the biological activity and functionality of the platelets produced from the human embryonic stem cells. Platelets produced from this in vitro platelet production system are functionally and resemble normal platelets in the human body. However, when produced by in vitro generation and maturation, the platelets produced will be readily distinguishable from human platelets derived from blood due to the fact that the platelets produced by this process will never have been exposed, at least as produced, to human blood.

I/We claim:

1. A method for the production of human platelets in vitro comprising the steps of
(a) culturing human embryonic stem cells under conditions which favor the differentiation of the cells into the hematopoietic lineage;
(b) culturing the cells of the hematopoietic lineage into megakaryocytes;
(c) culturing the megakaryocytes so that they will produce platelets; and
(d) recovering the platelets apart from the megakaryocytes.

2. A method as claimed in claim 1 wherein the step (a) is performed by encouraging the formation of embryoid bodies and then selectively recovering hematopoietic cells from the embryoid bodies.

3. A method as claimed in claim 1 wherein the step (a) is performed by co-culture of the human embryonic stem cells with stromal cells.

4. A method as claimed in claim 1 wherein the step (b) is performed by culturing the cells from step (a) in a medium including thrombopoeitin, interleukin 3, interleukin 6, interleukin 11, FLT-3 and stem cell factor

5. A method as claimed in claim 1 wherein the megakaryocytes are positive for CD41, CD42a, CD42b, CD61, CD38, CD45 (weak) and CD62P, but negative for CD34, CD117, beta-2-microglobulin, and HLA-DR

6. A method as claimed in claim 1 further comprising exposing the culture of megakaryocytes to nitric oxide to encourage the budding of platelets.

7. A method as claimed in claim 6 wherein the nitric oxide is produced by adding GMSO to the culture.

8. A method claimed in claim 1 wherein the platelets are separated from the megakaryocytes by low speed centrifugation.

9. Human platelets produced by in vitro culture using the process of claim 1.

10. The human platelets of claim 9 wherein the platelets are free of immunoglobulin molecules.

11. Human platelets produced in in vitro culture, the platelets being biologically active to initiate clotting, the platelets being substantially free of and never having been exposed to blood antigens, lymphocytes and constituents of human blood plasma.

12. An aliquot of human platelets comprising functional human platelets produced in vitro and substantially free of immunoglobulins and substantially free of ABO and Rh antigens.

13. An aliquot of human platelets produced by in vitro culture, the platelets being biologically active to initiate clotting in vivo, the platelets carried in a medium containing no human serum or plasma.
